(19) United States
(12) Patent Application Publication
(10) Pub. No.: US 2009/0182053 A1
(43) Pub. Date: Jul. 16, 2009

(54) FENOFIBRIC ACID AMORPHOUS DISPERSION; METHOD OF MAKING; AND METHOD OF USE THEREOF

(76) Inventors: Tong Sun, Marlton, NJ (US); Shawn Watson, Cherry Hill, NJ (US); Heidi A. Pazamickas, Albany, NY (US); Joseph Forth, Albany, NY (US)

Correspondence Address: CANTOR COLBURN, LLP, 20 Church Street, 22nd Floor, Hartford, CT, 06103, US

(21) Appl. No.: 12/335,698

(22) Filed: Dec. 16, 2008

Related U.S. Application Data
(60) Provisional application No. 61/014,964, filed on Dec. 19, 2007.

(51) Int. Cl. A61K 31/192; A61P 3/06

(52) U.S. Cl. 514/571

(57) ABSTRACT

Disclosed are fenofibric acid amorphous dispersions containing amorphous fenofibric acid and an amorphous dispersion excipient. The amorphous dispersions can be prepared by e.g., spray drying techniques and formulated into pharmaceutical products.

### CROSS REFERENCE TO RELATED APPLICATION

This application claims the benefit of U.S. Provisional Application Ser. No. 61/014,964 filed Dec. 19, 2007, which is hereby incorporated by reference in its entirety.

### BACKGROUND

Fenofibrate, 2-[4-(4-chlorobenzoyl)phenoxy]-2-methyl-propanoic acid, 1-methylethyl ester, is used in the treatment of endogenous hyperlipidaemias, hypercholesterolaemias and hypertriglyceridaemias in adults. Fenofibric acid, the active metabolite of fenofibrate, produces reductions in total cholesterol, LDL cholesterol, apolipoprotein B, total triglycerides and triglyceride rich lipoprotein (VLDL) in treated patients. Also, treatment with fenofibrate results in increases in high-density lipoprotein (HDL) and apoproteins apoAI and apoAII. Prolonged treatment with fenofibrate at the rate of about 300 to about 400 mg per day makes it possible to obtain a reduction in total cholesterol of about 20 to about 25% and a reduction in the levels of triglycerides of about 40 to about 50%.

Fenofibrate is not soluble in water, which limits its absorption in the gastrointestinal (GI) tract. To remedy this problem, research groups have tried a multitude of strategies including, for example, formulations comprising reduced sized fenofibrate, the combination of fenofibrate and vitamin E, the use of diethylene glycol monoethyl ether (DGME) as solubilizer, and the combination of fenofibrate with one or more polyglycolyzed glycerides.

There accordingly remains the need for soluble forms of fenofibrate as well as improved lipid regulating agent dosage forms.

### SUMMARY

In one embodiment, an amorphous dispersion comprises, fenofibric acid or a pharmaceutically acceptable salt thereof and an amorphous dispersion excipient; wherein the fenofibric acid or a pharmaceutically acceptable salt thereof within the dispersion is in substantially amorphous form; and wherein the dispersion is free of an enteric polymer.

In another embodiment, a method of preparing an amorphous dispersion comprises forming a mixture comprising fenofibric acid or a pharmaceutically acceptable salt thereof, an amorphous dispersion excipient, and a solvent; and spray drying the mixture to result in an amorphous dispersion, wherein the fenofibric acid or a pharmaceutically acceptable salt thereof within the dispersion is in substantially amorphous form.

In yet another embodiment, a composition comprises a spray dried amorphous fenofibric acid dispersion comprising fenofibric acid or a pharmaceutically acceptable salt thereof and an amorphous dispersion excipient, wherein the fenofibric acid or a pharmaceutically acceptable salt thereof within the dispersion is in substantially amorphous form; and a pharmaceutically acceptable excipient.

In still another embodiment, a method of treating a patient comprises administering to a patient in need thereof the amorphous dispersion comprising fenofibric acid or a pharmaceutically acceptable salt thereof or a composition comprising the amorphous dispersion.

These and other embodiments, advantages and features of the present invention become clear when detailed description and examples are provided in subsequent sections.

### BRIEF DESCRIPTION OF DRAWINGS

FIG. 1 illustrates an XRPD pattern of spray dried fenofibric acid and Plasdone K-25.

FIG. 2 illustrates an XRPD pattern of spray dried fenofibric acid and HPMC.

FIG. 3 illustrates an XRPD pattern of spray dried fenofibric acid and Crospovidone XL-10/Copovidone S-630.

### DETAILED DESCRIPTION

Disclosed herein are amorphous dispersions of fenofibric acid (2-[4-(4-chlorobenzoyl)phenoxy]-2-methyl-propanoic acid) or a pharmaceutically acceptable fenofibric acid salt thereof in combination with an amorphous dispersion excipient, wherein the fenofibric acid or a pharmaceutically acceptable salt thereof within the dispersion is in substantially amorphous form. The fenofibric acid or salt thereof in amorphous form allows an increase in the bioavailability of the active agent when orally dosed to a patient. When formulated into dosage forms, the amorphous fenofibric acid dispersion exhibits a quicker dissolution rate when analyze in in vitro dissolution tests as compared to dosage forms prepared with crystalline fenofibric acid.

“Substantially amorphous form” means no recognizable characteristic crystalline fenofibric acid or fenofibric acid salt peaks or excipient peaks are present in an X-ray powder diffraction pattern of the material.

Fenofibric acid may be used in the amorphous dispersion as the free acid or in a pharmaceutically acceptable salt form. “Pharmaceutically acceptable salts” include derivatives of fenofibric acid, wherein the fenofibric acid is modified by making base addition salts thereof, and further refers to pharmaceutically acceptable solvates, including hydrates, of such salts. Examples of pharmaceutically acceptable salts include, but are not limited to, alkali or organic addition salts of acidic residues; and the like. For example, acceptable inorganic salts include metal salts such as sodium salt, potassium salt, cesium salt, and the like; and alkaline earth metal salts, such as calcium salt, magnesium salt, and the like; examples of organic salts include alkyl amine salts such as ethanolamine, diethanolamine, methylamine, dimethylamine, trimethylamine, triethylamine, ethylamine, diethylamine, ethylenediamine, piperazine, and the like; and combinations comprising one or more of the foregoing salts.

The amorphous dispersion excipient can be any pharmaceutically acceptable excipient that once coprocessed with fenofibric acid or a salt thereof, functions to maintain the fenofibric acid/salt in amorphous form. The amorphous dispersion excipient to be combined with the fenofibric acid can be crystalline or amorphous. Exemplary amorphous dispersion excipients include cellulosic polymers such as an alkylcelluloses (e.g., methylcellulose, ethylcellulose, and the like); modified celluloses such as a carboxymethylcellulose, a hydroxypropyl methyl cellulose, a crosslinked sodium carboxymethylcellulose, a hydroxyl alkylcellulose (e.g., hydroxypropylcellulose); a polyvinylpyrrolidone; a crosslinked homopolymer of N-vinyl-2-pyrrolidone; a polyvinylpyrrolidone-vinyl acetate copolymer; a polyvinyl alcohol; a polysaccharide; a mono or disaccharide (e.g., lactose); a sugar alcohol; or a combination comprising at least one of the foregoing amorphous dispersion excipients.

The amount of fenofibric acid or salt thereof to the total amount of amorphous dispersion excipient can be about 1:20 to about 5:1, specifically about 1:10 to about 1:1, and yet more specifically about 1:5 to about 1:3 wt/wt. The amorphous dispersion excipient can be a single excipient or a combination of two or more excipients. In a binary excipient system, exemplary ratios of first amorphous dispersion excipient to second amorphous dispersion excipient can be about 1:20 to about 20:1, specifically about 1:10 to about 10:1, and yet more specifically about 1:5 to about 5:1 wt/wt.

In addition to the amorphous dispersion excipient, the amorphous dispersion can optionally further comprise other excipients to aid in processing such as surfactants, plasticizers, fillers, and the like.

In one embodiment, the amorphous dispersion is free of enteric polymers. “Enteric polymer” means a polymer which is preferentially soluble in the less acid environment of the intestine relative to the more acid environment of the stomach.

Determination of the extent of conversion of the crystalline form of fenofibric acid to an amorphous form can be determined using analytical techniques known in the art, including x-ray diffraction analysis, differential scanning calorimetry, optical microscopy, and the like.

The fenofibric acid/salt amorphous dispersion can be prepared by forming a mixture of fenofibric acid/salt and amorphous dispersion excipient and removing the solvent to form the amorphous dispersion.

Suitable solvents for preparing the amorphous dispersion include those that do not adversely affect the stability of the fenofibric acid, and are preferably inert. Suitable solvents may be organic, aqueous, or a mixture thereof. Suitable organic solvents may be aliphatic alcohols such as methanol, ethanol, n-propanol, and isopropanol; aliphatic ketones such as acetone and methyl ethyl ketone; aliphatic carboxylic esters such as ethyl acetate; aromatic hydrocarbons such as toluene and xylene; aliphatic hydrocarbons such as hexane; aliphatic nitriles such as acetonitrile; chlorinated hydrocarbons such as dichloromethane; aliphatic sulfoxides such as dimethyl sulfoxide; and the like, as well as mixtures comprising at least one of the foregoing organic solvents. Specifically aqueous solvents are used, that is, a solvent comprising water and/or a water-miscible organic solvent such as a lower alcohol, acetonitrile, tetrahydrofuran, dimethylacetamide, dimethyl formamide, and the like.

As used herein “solvent” can include a combination of two or more solvents.

Choice and amount of solvent can be determined by one of ordinary skill in the art without undue experimentation. The solvent is chosen such that the mixture contains fenofibric acid that is completely dissolved or substantially dissolved in the solvent. In an exemplary embodiment, the solvent comprises an aqueous solvent comprising about 1 to about 99 percent by weight water miscible organic solvent and about 99 to about 1 weight percent water, specifically about 10 to about 80 percent by weight water miscible organic solvent and about 90 to about 20 weight percent water, and more specifically about 20 to about 50 percent by weight water miscible organic solvent and about 80 to about 50 weight percent water.

The process of preparing fenofibric acid/salt amorphous dispersion involves dissolving or suspending the fenofibric acid/salt and the amorphous dispersion excipient in solvent, followed by removal of the solvent to leave a solid dispersion of amorphous fenofibric acid/salt and amorphous dispersion excipient. Such a process can include preparation of a separate solution or suspension of fenofibric acid/salt and solvent which is then blended with a solution or suspension of amorphous dispersion excipient and solvent, followed by removal of the solvent. Other approaches include dissolving the fenofibric acid/salt and the amorphous dispersion excipient in the same solution.

Optionally, to aid in dissolution of the fenofibric acid/salt or amorphous dispersion excipient within the solvent, the solution or suspension can be heated or sonicated. The solution or suspension can be heated up to about 150° C., specifically about 30° C. to about 100° C., more specifically about 40 to about 70° C., and yet more specifically about 45° C. to about 50° C.

In one embodiment, to improve the stability of the amorphous dispersion, a true solution of fenofibric acid/salt, amorphous dispersion excipient, and solvent can be prepared prior to removal of the solvent. Specifically, the solution contains no undissolved solids present in solution. Optionally, the solution of fenofibric acid/salt, amorphous dispersion excipient, and solvent can be filtered to remove any undissolved solids, solid impurities and the like prior to removal of the solvent. Any filtration system and filtration techniques known in the art can be used.

In another embodiment, either a cloudy solution or a suspension of fenofibric acid/salt, amorphous dispersion excipient, and solvent can be prepared prior to removal of the solvent. Specifically, the fenofibric acid/salt is substantially dissolved in the mixture and the solids present can be due to the excipient, specifically an amorphous excipient.

The solvent can be removed by any variety of methods such as evaporation optionally under reduced pressure or under heat, precipitation by a non-solvent, freeze drying, spray drying, and the like; specifically by spray drying techniques.

The mixture of fenofibric acid, amorphous dispersion excipient, and solvent can by spray dried using techniques and equipment known in the art. The spray drier atomizes the solution in a stream of air or other gas resulting in rapid evaporation of the solvent leaving behind a dispersion of amorphous fenofibric acid and amorphous dispersion excipient.

Also disclosed herein are pharmaceutical compositions comprising the fenofibric acid/salt amorphous dispersion.

Solid dosage forms for oral administration include, but are not limited to, capsules, tablets, powders, and granules. In such solid dosage forms, the amorphous dispersion may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules and tablets, the dosage forms may also comprise buffering agents.

By “oral dosage form” is meant to include a unit dosage form for oral administration. An oral dosage form may optionally comprise a plurality of subunits such as, for example, microcapsules or microtablets. Multiple subunits may be packaged for administration in a single dose.

By “subunit” is meant to include a composition, mixture, particle, pellet, etc., that can provide an oral dosage form alone or when combined with other subunits.

The compositions can be immediate-release forms or controlled-release forms.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate-release and controlled-release characteristics, for example, a combination of immediate-release pellets and controlled-release pellets. The immediate-release portion of a combination dosage form may be referred to as a loading dose.

Certain compositions described herein may be “coated”. The coating may be a suitable coating, such as, a functional or a non-functional coating, or multiple functional and/or non-functional coatings. By “functional coating” is meant to include a coating that modifies the release properties of the total composition, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

In one embodiment, the composition comprising the fenofibric acid/salt amorphous dispersion is bioequivalent to the reference drug Tricor®. “Reference drug” means a fenofibrate product as described in U.S. Federal Food and Drug Administration's New Drug Application No. 021656 approved on Nov. 5, 2004 as provided in the U.S. Federal Food and Drug Administration's Orange Book, Approved Drug Products with Therapeutic Equivalence Evaluations. Tricor® is a fenofibrate tablet product at a strength of 48 mg or 145 mg. Tricor®, 145 mg strength is the “reference listed drug” under 21 CFR 314.94(a)(3)), i.e., the listed drug identified by FDA as the drug product upon which an applicant relies in seeking approval of its ANDA.

“Bioequivalence” means the absence of a significant difference in the rate and extent to which the active agent or surrogate marker for the active agent in pharmaceutical equivalents or pharmaceutical alternatives becomes available at the site of action when administered in an appropriately designed study.

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS-GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE”DHHS, FDA, CDER, January 2001, both of which are incorporated herein in their entirety.

In another embodiment, bioequivalence is determined according to the European Medicines Agency (EMEA) document “Note for Guidance on the Investigation of Bioavailability and Bioequivalence”, issued Jul. 26, 2001, available from EMEA.

In one embodiment, a fenofibric acid/salt amorphous dispersion composition is bioequivalent to tablet formulations commercially available in the United States, for example the reference drug of NDA # 021656.

In one embodiment, a fenofibric acid/salt amorphous dispersion composition exhibits a ratio of a geometric mean of logarithmic transformed AUC_{0-∞} of the composition to a geometric mean of logarithmic transformed AUC_{0-∞} of fenofibrate reference drug of about 0.80 to about 1.25. In another embodiment, a fenofibric acid/salt amorphous dispersion composition exhibits a ratio of a geometric mean of logarithmic transformed AUC₀₋ₜ of the composition to a geometric mean of logarithmic transformed AUC₀₋ₜ of fenofibrate reference drug of about 0.80 to about 1.25. In yet another embodiment, a fenofibric acid/salt amorphous dispersion composition exhibits a ratio of a geometric mean of logarithmic transformed Cₘₐₓ of the composition to a geometric mean of logarithmic transformed Cₘₐₓ of fenofibrate reference drug of about 0.70 to about 1.43. In yet another embodiment, a fenofibric acid/salt amorphous dispersion composition exhibits a ratio of a geometric mean of logarithmic transformed Cₘₐₓ of the composition to a geometric mean of logarithmic transformed Cₘₐₓ of fenofibrate reference drug of about 0.80 to about 1.25.

In an embodiment, bioequivalence of a fenofibric acid/salt amorphous dispersion composition to a reference drug is determined by an in vivo pharmacokinetic study to determine a pharmacokinetic parameter for the composition. Specifically, bioequivalence can be determined by an in vivo pharmacokinetic study comparing a pharmacokinetic parameter for the two compositions. A pharmacokinetic parameter for the composition or the reference drug can be measured in a single or multiple dose bioequivalence study using a replicate or a nonreplicate design. For example, the pharmacokinetic parameters for the composition of the present invention and for a reference drug can be measured in a single dose pharmacokinetic study using a two-period, two-sequence crossover design. Alternately, a four-period, replicate design crossover study may also be used. Single doses of the test composition and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

Under U.S. FDA guidelines, two products (e.g. an inventive composition and Tricor®) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

To show bioequivalency between two compounds pursuant to Europe's EMEA guidelines, the 90% CI limits for a ratio of the geometric mean of logarithmic transformed AUC_{0-∞} and AUC₀₋ₜ for the two products are about 0.80 to about 1.25. The 90% CI limits for a ratio of the geometric mean of logarithmic transformed Cₘₐₓ for the two products can have a wider acceptance range when justified by safety and efficacy considerations. For example the acceptance range can be about 0.70 to about 1.43, specifically about 0.75 to about 1.33, and more specifically about 0.80 to about 1.25.

In an embodiment, in a given experiment, a fenofibric acid/salt amorphous dispersion composition is considered to be bioequivalent to Tricor® if both the Test/Reference ratio for the geometric mean of logarithmic transformed AUC_{0-∞}, AUC₀₋ₜ, or Cₘₐₓ ratio along with its corresponding lower and upper 90% CI limits are within a lower limit of about 0.80 and an upper limit of about 1.25. Thus, for direct comparison between a fenofibric acid/salt amorphous dispersion composition and Tricor®, it is sometimes preferred to determine the pharmacokinetic parameters for the fenofibric acid/salt amorphous dispersion composition and Tricor® side-by-side in the same pharmacokinetic study.

In other embodiments, the single dose pharmacokinetic study is conducted between the fenofibric acid/salt amorphous dispersion composition and the reference listed drug using the strength specified by the FDA in APPROVED DRUG PRODUCTS WITH THERAPEUTIC EQUIVALENCE EVALUATIONS(ORANGE BOOK).

In one embodiment, the fenofibric acid/salt dispersion is combined or administered with a second active agent. In particular, those agents with an action like that of fenofibric acid, e.g., other lipid regulating agents, such as further fibrates, e.g., bezafibrate, ciprofibrate and gemfibrocil, or statins, e.g., lovastatin, mevinolin, pravastatin, fluvastatin, atorvastatin, itavastatin, mevastatin, rosuvastatin, velostatin, synvinolin, simvastatin, cerivastatin and numerous others mentioned in, for instance, WO 02/067901 may be employed.

In another embodiment, fenofibric acid/salt amorphous dispersion is combined with an agent suitable for the treatment of high blood pressure such as, for example, diuretics (chlorthalidone, furosemide, hydrochlorothiazide, indapamide, metolazone, amiloride hydrochloride, spironolactone, and triamterene), beta-blockers (acebutolol, atenolol, betaxolol, bisoprolol fumarate, carteolol hydrochloride, metoprolol tartrate, metoprolol succinate, nadolol, penbutolol sulfate, pindolol, propranolol hydrochloride, and timolol maleate), sympathetic nerve inhibitors (guanadrel, guanethidine monosulfate, and reserpine), vasodilators (hydralazine hydrocholoride and minoxidil), angiotensin-converting enzyme (ACE) inhibitors (benazepril hydrochloride, captopril, enalapril maleate, fosinopril sodium, lisinopril, moexipril, quinapril hydrochloride, ramipril and trandolapril), angiotensin II receptor blockers (candesartan, irbesarten, losartin potassium, and valsartan) and the calcium antagonists (calcium channel blockers, e.g., amlodipine besylate, diltiazem hydrochloride, felodipine, isradipine, nicardipine, nifedipine, nisoldipine, and verapamil hydrochloride).

In another embodiment, fenofibric acid/salt amorphous dispersion is combined with aspirin or other blood thinning agents such as Coumadin® (warfarin), Dicumarol® (dicumarol), and Miradon® (anisinidione).

In yet another embodiment, fenofibric acid/salt amorphous dispersion is combined with a calcium supplement, such as, for example, calcium citrate. A method of improving the absorption of fenofibric acid comprises coadministering with a calcium supplement, i.e., calcium citrate.

The fenofibric acid/salt amorphous dispersions and compositions prepared therefrom are useful in treating conditions such as hypercholesterolemia, hypertriglyceridemia, cardiovascular disorders, coronary heart disease, and peripheral vascular disease (including symptomatic carotid artery disease). The fenofibric acid/salt amorphous dispersions compositions can be used as adjunctive therapy to diet for the reduction of LDL-C, total-C, triglycerides, and Apo B in adult patients with primary hypercholesterolemia or mixed dyslipidemia (Fredrickson Types IIa and IIb). The fenofibric acid/salt amorphous dispersions can also be used as adjunctive therapy to diet for treatment of adult patients with hypertriglyceridemia (Fredrickson Types IV and V hyperlipidemia). Markedly elevated levels of serum tryglycerides (e.g., >2000 mg/dL) may increase the risk of developing pancreatitis. The fenofibric acid/salt amorphous dispersions can also be used for other indications where lipid regulating agents are typically used.

The following examples further illustrate the invention but, of course, should not be construed as in any way limiting its scope.

### EXAMPLES

### Example 1

### Preparation of Fenofibric Acid Amorphous Dispersions

Several fenofibric acid amorphous dispersions are prepared by spray drying solutions of fenofibric acid and an amorphous dispersion excipient in an aqueous solution as shown in Table 1. A quantity of fenofibric acid is added to about 400 milliliters (ml) of isopropanol (IPA) and sonicated to ensure complete dissolution of the fenofibric acid. The volume of the solution is then increased to 2000 ml by the addition of water. The resulting solution is heated in a 40° C. oven overnight and then heated to approximately 50° C. to form a clear solution. A quantity of an amorphous dispersion excipient is added to the fenofibric acid and IPA/water solution and stirred for one hour to form a clear solution. The example with HPMCP did not form a clear solution.

TABLE 1

| | | Excipient | | |
|---|---|---|---|---|
| | | amount | Fenofibric | |
| | Amorphous Dispersion Excipient | (mg) | acid (mg) | XRPD Result |
| A | Polyvinylpyrrolidone (Plasdone K-25) | 251.57 | 253.34 | amorphous |
| B | Polyvinylpyrrolidone (Plasdone K-25) | 351.26 | 150.63 | amorphous |
| C | Polyvinylpyrrolidone (Plasdone K-25) | 154.82 | 351.28 | crystalline |
| D | Hydroxypropyl methyl cellulose (HPMC) | 249.63 | 249.15 | amorphous |
| E | Hydroxypropyl methyl cellulose (HPMC) | 356.42 | 152.34 | amorphous |
| F | Hydroxypropyl methyl cellulose (HPMC) | 152.63 | 349.37 | crystalline |
| G | Crosslinked homopolymer of N-vinyl-2- | 175.23/75.84 | 251.17 | amorphous |
| | pyrrolidone (Crospovidone XL-10)/ | | | |
| | polyvinylpyrrolidone-vinyl acetate copolymer | | | |
| | (Copovidone S-630) | | | |
| H | Crosslinked homopolymer of N-vinyl-2- | 200.13/150.46 | 151.75 | amorphous |
| | pyrrolidone (Crospovidone XL-10)/ | | | |
| | polyvinylpyrrolidone-vinyl acetate copolymer | | | |
| | (Copovidone S-630) | | | |
| I | Crosslinked homopolymer of N-vinyl-2- | 91.43/60.45 | 352.41 | crystalline |
| | pyrrolidone (Crospovidone XL-10)/ | | | |
| | polyvinylpyrrolidone-vinyl acetate copolymer | | | |
| | (Copovidone S-630) | | | |
| J | Hydroxypropyl methyl cellulose phthalate | 252.36 | 252.79 | amorphous |
| | (HPMCP) | | | |

The solutions of fenofibric acid and amorphous dispersion excipient are spray dried according to the conditions in Table 2.

TABLE 2

| Model | Buchi Mini Spray Dryer B-290 |
|---|---|
| Inlet temperature | 105° C. |
| Outlet temperature | ~53° C. |
| Aspirator | 100% |
| Pump | 25% |
| Air Flow | 30-35 mm |
| Nozzle size | 1.5 mm |

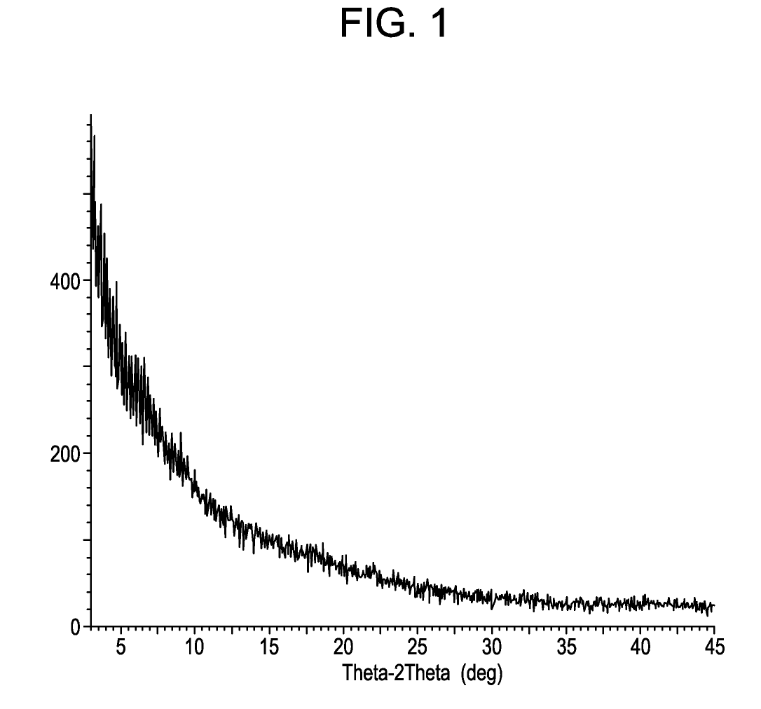

The isolated solids are analyzed by x-ray powder diffraction (XRPD) and optical microscopy. XRPD patterns (FIGS. 1-3, formulations A, D, and G, respectively) indicate the isolated solids of the HPMC, Plasdone K-25, and Crospovidone XL-10/Copovidone S-630 are amorphous. Optical microscopy analysis indicates residual crystalline material in the HPMC mixture. Only small amounts of residual crystallinity are detected via microscopy in the Plasdone K-25 and Crospovidone XL-10/Copovidone S-630 examples.

### Example 2

### Interaction Time

The amount of interaction/stirring time is assessed to determine the impact on amorphous material generation via spray drying. Portions of the solutions prepared in Example 1 are allowed to stand for 72 hours resulting in the slight precipitation of fenofibric acid from each solution. Each solution is reheated to approximately 50° C. and spray dried according to the conditions in Table 2. The isolated solids are analyzed by XRPD and optical microscopy. Only small amounts of residual crystallinity are detected via microscopy in the Plasdone K-25 and Crospovidone XL-10/Copovidone S-630 examples. The HPMC sample exhibited increased crystallinity in both the XRPD and optical microscopy analyses.

### Example 3

### Accelerated Stability Study

Six excipient combinations are prepared to examine the stability of the prepared amorphous dispersions (Table 3). About 200 mg of each material is placed in 4 dram amber vials, covered with foil, and placed in a controlled 40° C./75% relative humidity chamber for one week. The 30:70 fenofibric acid:Plasdone K-25 maintained its amorphous form after the accelerated study. High performance liquid chromatography on all the samples revealed no significant degradation of the fenofibric acid.

TABLE 3

| | | | XRPD | % Purity |
|---|---|---|---|---|
| | XRPD | % Purity | (t = | (t = |
| Mixture | (initial) | (initial) | 1 week) | 1 week) |
| 30:70 Fenofibric | Amorphous | 98.8 | Amorphous | 99.4 |
| acid:Plasdone K-25 | | | | |
| 50:50 Fenofibric | Amorphous | 98.3 | Crystalline | 99.4 |
| acid:Plasdone K-25 | | | | |
| 5:3:2 Fenofibric | Amorphous | 98.9 | Crystalline | 98.4 |
| acid:Crospovidone | | | | |
| XL-10:Copovidone | | | | |
| S-630 | | | | |
| 3:5:2 Fenofibric | Amorphous | 99.5 | Slightly | 99.9 |
| acid:Crospovidone | | | Crystalline | |
| XL-10:Copovidone | | | | |
| S-630 | | | | |
| 50:50 Fenofibric | Amorphous | 98.4 | Crystalline | 99.8 |
| acid:HPMC | | | | |
| 30:70 Fenofibric | Amorphous | 99.1 | Crystalline | 98.9 |
| acid:HPMC | | | | |

### Example 4

### Short Term Stability Study

Six excipient combinations are prepared to examine the stability of the amorphous dispersions (Table 4). Sample solutions are prepared by adding fenofibric acid to a clean 4000 ml bottle with 800 ml isopropanol. The solutions are sonicated and each mixture is then brought up to a total volume of 4000 ml with water. The solutions are then heated to about 50° C. to obtain a clear solution and the excipient(s) is added. The solutions are spray dried while being stirred. The spray drying is performed on a Buchi Mini Spray Dryer B-290 with an inlet temperature of 165° C., aspirator at 100%, pump at 25%, air flow at 30-35 mm and a nozzle size of 1.5 mm.

About 200 mg of each material is placed in capped, sealed 4 dram amber vials, and placed in controlled 25° C./60% relative humidity and 40° C./75% relative humidity chambers. The materials are tested after one week and then three weeks to monitor for form changes. All materials except the 50:50 fenofibric acid:HMPC remained amorphous after the first week. The 30:70 fenofibric acid:Plasdone K-25 remained fully amorphous after three weeks.

TABLE 4

| | XRPD | XRPD (t = 1 week) | | XRPD (t = 3 week) | |
|---|---|---|---|---|---|
| Mixture | (initial) | 25 C./60% RH | 40 C./75% RH | 25 C./60% RH | 40 C./75% RH |
| 30:70 Fenofibric | Amorphous | Amorphous | Amorphous | Amorphous | Amorphous |
| acid:Plasdone K-25 | | | | | |
| 50:50 Fenofibric | Amorphous | Amorphous | Slightly | Slightly | Crystalline |
| acid:Plasdone K-25 | | | Crystalline | Crystalline | |
| 5:3:2 Fenofibric | Amorphous | Amorphous | Slightly | Crystalline | Crystalline |
| acid:Crospovidone | | | Crystalline | | |
| XL-10:Copovidone | | | | | |
| S-630 | | | | | |
| 3:5:2 Fenofibric | Amorphous | Amorphous | Slightly | Slightly | Crystalline |
| acid:Crospovidone | | | Crystalline | Crystalline | |
| XL-10:Copovidone | | | | | |
| S-630 | | | | | |
| 30:70 Fenofibric | Amorphous | Amorphous | Amorphous | Slightly | Slightly |
| acid:HPMC | | | | Crystalline | Crystalline |
| 50:50 Fenofibric | Amorphous | Slightly | Crystalline | Crystalline | Crystalline |
| acid:HPMC | | Crystalline | | | |

The terms “comprising”, “having”, “including”, and “containing” are to be construed as open-ended terms (i.e., meaning “including, but not limited to”). The terms “a” and “an” do not denote a limitation of quantity, but rather denote the presence of at least one of the referenced item. The term “or” means “and/or”. The endpoints of all ranges directed to the same component or property are inclusive and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An amorphous dispersion, comprising:
a) fenofibric acid or a pharmaceutically acceptable salt thereof and an amorphous dispersion excipient,
wherein the fenofibric acid or a pharmaceutically acceptable salt thereof within the dispersion is in substantially amorphous form, and
wherein the dispersion is free of an enteric polymer; or
b) a spray dried combination of fenofibric acid or a pharmaceutically acceptable salt thereof and an amorphous dispersion excipient;
wherein the fenofibric acid or a pharmaceutically acceptable salt thereof within the dispersion is in substantially amorphous form.

2. The dispersion of claim 1, comprising fenofibric acid.

3. The dispersion of claim 1, wherein the dispersion is a spray dried combination of
fenofibric acid or a pharmaceutically acceptable salt thereof and
an amorphous dispersion excipient.

4. The dispersion of claim 1, comprising a weight ratio of fenofibric acid or a pharmaceutically acceptable salt thereof to amorphous dispersion excipient of about 1:20 to about 5:1.

5. The dispersion of claim 1, comprising a weight ratio of fenofibric acid or a pharmaceutically acceptable salt thereof to amorphous dispersion excipient of about 1:10 to about 1:1.

6. The dispersion of claim 1, wherein the amorphous dispersion excipient is a cellulosic polymer, a modified cellulose, a polyvinylpyrrolidone, a crosslinked homopolymer of N-vinyl-2-pyrrolidone, a polyvinylpyrrolidone-vinyl acetate copolymer, a polyvinyl alcohol, a polysaccharide, a mono or disaccharide, a sugar alcohol, or a combination thereof.

7. The dispersion of claim 1, wherein the amorphous dispersion excipient is a hydroxypropyl methyl cellulose, a polyvinylpyrrolidone, a crosslinked homopolymer of N-vinyl-2-pyrrolidone, a polyvinylpyrrolidone-vinyl acetate copolymer, a polyvinyl alcohol, or a combination thereof.

8. The dispersion of claim 1, wherein the amorphous dispersion excipient is a polyvinylpyrrolidone or a combination of a crosslinked homopolymer of N-vinyl-2-pyrrolidone and a polyvinylpyrrolidone-vinyl acetate copolymer.

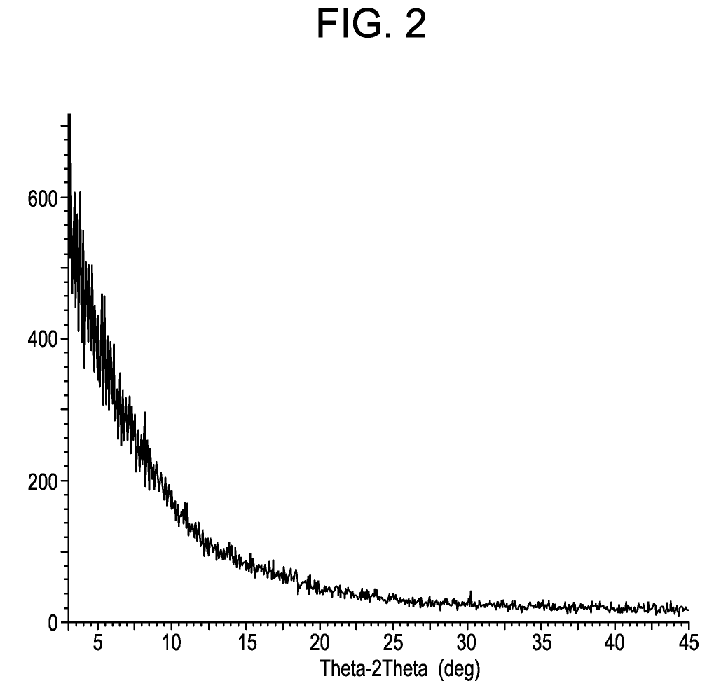

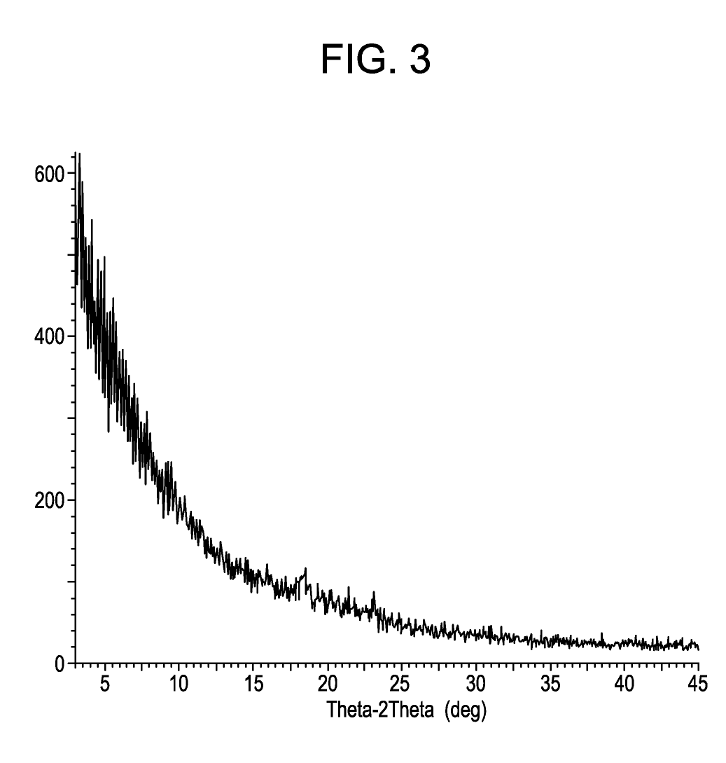

9. The dispersion of claim 1 exhibiting an X-ray powder diffraction pattern substantially similar to FIG. 1, FIG. 2, or FIG. 3.

10. A method of preparing an amorphous dispersion, comprising:
forming a mixture comprising fenofibric acid or a pharmaceutically acceptable salt thereof, an amorphous dispersion excipient, and a solvent; and
spray drying the mixture to result in an amorphous dispersion,
wherein the fenofibric acid or a pharmaceutically acceptable salt thereof within the dispersion is in substantially amorphous form.

11. The method of claim 10, wherein the solvent is an aqueous solvent.

12. The method of claim 11, wherein the aqueous solvent is water or a combination of water and a water miscible organic solvent.

13. The method of claim 10, wherein the dispersion is free of an enteric polymer.

14. A composition, comprising:
a spray dried amorphous fenofibric acid dispersion comprising fenofibric acid or a pharmaceutically acceptable salt thereof and an amorphous dispersion excipient,
wherein the fenofibric acid or a pharmaceutically acceptable salt thereof within the dispersion is in substantially amorphous form; and
a pharmaceutically acceptable excipient.

15. The composition of claim 14, wherein the composition is a solid oral dosage formulation.

16. The composition of claim 14, wherein the composition is bioequivalent to a reference drug according to NDA #021656.

17. The composition of claim 16, wherein the 90% confidence limits of a ratio of a geometric mean of logarithmic transformed AUC_{0-∞} of the composition to a geometric mean of logarithmic transformed AUC_{0-∞} of the reference drug is about 0.80 to about 1.25.

18. The composition of claim 16, wherein the 90% confidence limits of a ratio of a geometric mean of logarithmic transformed AUC₀₋ₜ of the composition to a geometric mean of logarithmic transformed AUC₀₋ₜ of the reference drug is about 0.80 to about 1.25.

19. The composition of claim 16, wherein the 90% confidence limits of a ratio of a geometric mean of logarithmic transformed Cₘₐₓ of the composition to a geometric mean of logarithmic transformed Cₘₐₓ of the reference drug is about 0.7 to about 1.43.

20. The composition of claim 16, wherein the 90% confidence limits of a ratio of a geometric mean of logarithmic transformed Cₘₐₓ of the composition to a geometric mean of logarithmic transformed Cₘₐₓ of the reference drug is about 0.8 to about 1.25.

21. A method of treating a patient, comprising:
administering to a patient in need thereof the amorphous dispersion of claim 1.

22. A method of treating a patient, comprising:
administering to a patient in need thereof the composition of claim 14.
